(19) United States
(12) Patent Application Publication
(10) Pub. No.: US 2006/0189624 A1
(43) Pub. Date: Aug. 24, 2006

(54) Novel combination for treating sexual dysfunction

(75) Inventors: Erwin Bischoff, Wuppertal (DE); Hilmar Bischoff, Wuppertal (DE); Francois Giuliano, Saint Cloud (FR)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Aktiengesellschaft, Leverkusen (DE)

(21) Appl. No.: 11/347,741

(22) Filed: Feb. 3, 2006

Related U.S. Application Data
(63) Continuation of application No. 10/070,963, filed on Jun. 28, 2002, now abandoned.

(30) Foreign Application Priority Data
Sep. 15, 1999 (DE) 199 44 161.8

(51) Int. Cl. A61K 31/519; A61K 31/455; A61K 31/401; A61K 31/366; A61K 31/22

(52) U.S. Cl. 514/252.16; 514/262.1; 514/356; 514/423; 514/460; 514/548

(57) ABSTRACT

Disclosed is a combination preparation for the treatment of sexual dysfunction in men or women containing at least one active ingredient A and one active ingredient B as pharmaceutically active ingredients, whereby the active ingredient A is a PDE inhibitor, preferably a cGMP PDE inhibitor and the active ingredient B a lipid-reducing agent. Both the active ingredients A and B can be administered simultaneously or at alternate intervals, that is, as a functional unit or separated from each other.

### BACKGROUND OF THE INVENTION

The present invention relates to the area of sexual dysfunction in men and women.

The present invention relates in particular to a novel combination preparation for treating sexual dysfunction in men and women, in particular erectile dysfunction. The present invention thus also provides a novel combination therapy for the treatment of sexual dysfunction in men and women.

The present invention furthermore relates to the use of antilipernics for enhancing the activity of phosphodiesterase inhibitors (hereinbelow synonymously also referred to as “phosphodiesterase inhibitors” or “PDE inhibitors”) for treatment of sexual dysfunction.

From the prior art it is known that phosphodiesterase inhibitors—in particular those of subtype V, which are synonymously also referred to as “cGMP PDE inhibitors”—are suitable for treating sexual dysfunction, in particular for treating erectile dysfunction (see, for example, Molecular Pharmacology, 1999, 56, pages 124-130; Am. J. Physiol., Vol. 264, February 1993, pages H419-H422; The Journal of Urology, Vol. 147, pages 1650-1655 (June 1992); The New England Journal of Medicine, Vol. 326(2), pages 90-94 (9 Jan. 1992); International Journal of Impotence Research, 4, Suppl. 2, page 11 (1992); Drugs, News and Perspectives, 6(3), pages 150-156 (April 1993); Physiological Reviews 75, pages 191-236 (1995); Int. J. of Impotence 9, pages 17-26 (1997) and TIPS Reviews, Vol. 11, pages 150-155 (April 1990)).

Concerning the nomenclature of PDE inhibitors, reference is made to Beavo and Reifsnyder in Trends in Pharmacol. Sci. 1990. 11, pages 150-155 and to the article TIPS Reviews, Vol. 11, pages 150-155 (April 1990).

The reason for the effectiveness of PDE inhibitors, in particular cGMP PDE inhibitors (PDE V inhibitors), in the treatment of sexual, preferably erectile, dysfunction is that the neurotransmitter nitrogen monoxide NO, which is generated by the body following sexual stimulation, activates guanylate cyclase, which in turn converts GTP into cGMP, which for its part may then cause a relaxation of the corpus cavernosum, without the cGMP, which is responsible for the relaxation of the corpus cavernosum, being hydrolyzed by the enzyme phosphodiesterase V (PDE V) to give 5′GMP, since the activity of the enzyme PDE V is inhibited by the corresponding inhibitor. This mechanism is illustrated in the attached FIG. 1.

However, it has now been found that, in some patients, therapy of erectile dysfunction using PDE inhibitors, in particular cGMP PDE inhibitors, is unsuccessful or of only limited success. This group of patients comprises in particular patients having a disturbed endothelium function and/or metabolic disorders, such as, for example, hyperlipidemia (for example hypercholesterolemia), arteriosclerosis, diabetes (in particular of the diabetes mellitus type), and also heavy smokers and elderly patients. In these patients, the therapy of erectile dysfunction using customary doses results only in a considerably reduced effect, compared to other groups of patients which undergo therapy for erectile dysfunction but do not suffer from the abovementioned metabolic disorders.

However, it is exactly the abovementioned patient group having disturbed endothelium function and/or the abovementioned metabolic disorders which suffers more frequently than average from sexual dysfunction, in particular erectile dysfunction, which renders the customary treatment of this dysfunction with PDE inhibitors more difficult. To achieve a therapeutic effect, considerably higher doses of PDE inhibitors have to be administered to these problem patients, compared to other patient groups suffering from erectile dysfunction, but not from the metabolic disorders mentioned above. This, however, has—in addition to higher costs—the essential disadvantage that the side effects associated with the PDE inhibitor therapy are also increased by the factor of the higher dosage. These side effects include, for example, disturbed vision, in particular disturbed color vision and color perception, headaches and muscle aches. Side effects on the cardiovascular system, for example a lowering of the blood pressure, are also possible.

Surprisingly, the applicant has now found that the activity of PDE inhibitors, in particular PDE V inhibitors, in the therapy of sexual, preferably erectile, dysfunction—in particular in the groups of problem patients mentioned above—can be increased by combined administration of the PDE inhibitors with an antilipemic. In this manner, it is possible to avoid the disadvantages described above which are encountered in the customary treatment of erectile dysfunction when using PDE inhibitors alone, in particular in the problem patients mentioned above.

### DESCRIPTION OF THE DRAWING

FIG. 1 depicts the mechanism of action of PDE V inhibitors.

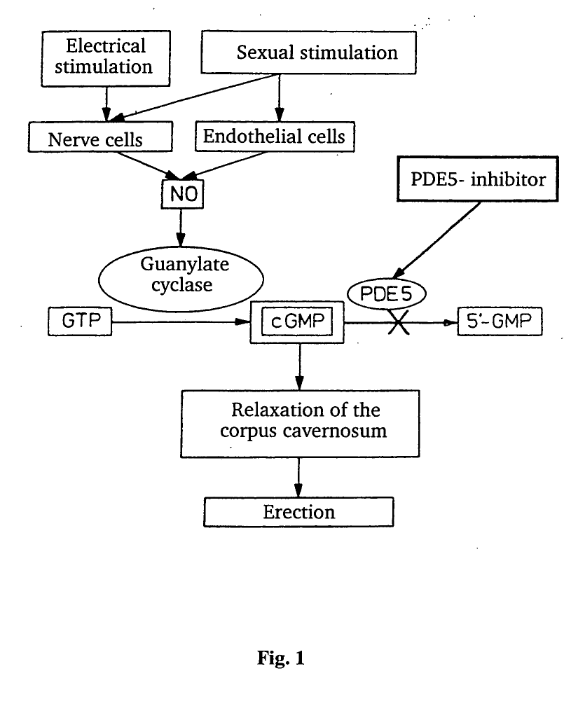

FIG. 2 depicts the synergistic enhancement of the cGMP PDE-inhibitory action.

### DESCRIPTION OF THE INVENTION

Accordingly, the present invention relates to a combination preparation comprising
as active compound component A at least one PDE inhibitor, preferably a PDE V inhibitor (cGMP PDE inhibitor); and
as active compound component B at least one antilipemic.

The combination preparation according to the invention is particularly suitable for therapy of sexual dysfunction, i.e. therapy of erectile dysfunction in men or sexual dysfunction in women.

At the same time, the combination preparation according to the invention allows a cotherapy of disturbed endothelium function (for example in elderly patients or heavy smokers) and/or a metabolic disorder, such as, for example, hyperlipidemia (for example hypercholesterolemia), arteriosclerosis or diabetes (in particular of the diabetes mellitus type).

The term “combination preparation”, as used for the purpose of the present invention, means that the two active compound components A and B can be used either simultaneously or else successively (i.e. separately).

Thus, according to the invention, the term “combination preparation” includes the ingredients A and B either in a functional unit, i.e. as a true combination (for example as a mixture, mix or blend), or else (spatially) separated, i.e. as a “kit-of-parts”.

Accordingly, the present invention also provides the use of antilipemics for enhancing the activity of PDE inhibitors (in particular PDE V inhibitors) in the therapy of sexual dysfunction, in particular erectile dysfunction.

The present invention furthermore provides a combination therapy for sexual dysfunction, in particular for erectile dysfunction, using a combination preparation which comprises at least one PDE inhibitor (in particular a PDE V inhibitor) and at least one antilipemic.

At the same time, the combination therapy according to the invention also allows a cotherapy of a disturbed endothelium function (for example in elderly patients or heavy smokers) and/or a metabolic disorder, such as, for example, hyperlipidemia (for example hypercholesterolemia), arteriosclerosis or diabetes (in particular of the diabetes mellitus type).

Using the combination according to the invention of PDE inhibitors and antilipemics—i.e., in other words, by applying the combination therapy according to the invention of sexual dysfunction using a combination of PDE inhibitor and antilipemic—it is possible to reduce the therapeutically required doses of PDE inhibitors in the abovementioned problem patients to customary doses like those administered to other patients suffering from erectile dysfunction but not from disturbed endothelium function or a metabolic disorder. However, according to the invention, it is also possible to reduce, by combined administration with an antilipemic, the dose of PDE inhibitor administered for the therapy of erectile dysfunction in patients who do not suffer from a specific metabolic disorder but who do suffer (for example because of old age) from erectile dysfunction.

As mentioned above, the combination according to the invention can be administered, i.e. the combination therapy according to the invention can be applied, by simultaneous administration of active compound components A and B. Here, the active compound components A and B can, as described above, be present either in a functional unit (i.e. as a true combination, such as, for example, as a mixture, a mix or a blend) or else (spatially) separated (i.e. as a “kit” or a “kit-of-parts”).

According to a preferred embodiment of the present invention, the active compound components A and B are administered separately, in particular successively.

This can take place, for example, by administering a daily dose of the antilipemic even a few days (for example about 1 week or else only 1-4 days) before administering the PDE inhibitor.

It is also possible to administer the PDE inhibitor in an ongoing therapy with antilipemics. Thus, according to the present invention it is possible, for example, to observe better successes in the therapy of erectile dysfunction using PDE inhibitors in men suffering from severe hypercholesterolemia in which the elevated cholesterol levels are already treated on a permanent basis using antilipemics.

However, surprisingly, similarly good therapeutic results are also observed in cases where the antilipemic is administered only a short time (for example a few days) before the administration of the PDE inhibitor. This is surprising and indicates an unforeseeable synergistic effect of the combination according to the invention, since in the patients treated in this manner, suffering, for example, from arteriosclerosis, there are not yet any changes in their symptoms, owing to the short duration of the therapy with the antilipemic.

According to a preferred embodiment of the present invention, the active compound components A and B of the combination preparation according to the invention are thus administered successively, with the antilipemic preferably being administered first, i.e. before the administration of the PDE inhibitor. This can be achieved by simply administering the antilipemic a short time before the administration of the PDE inhibitor, i.e. several times over a number of days or else only once a few hours beforehand, or else by administering the PDE inhibitor in an ongoing therapy with an antilipemic. Thus, in the latter case, the administration of the antilipemic can be continued both before and concomitantly with the administration of the PDE inhibitor.

Without wishing to adhere to a certain theory, the improved PDE-inhibitory action of the PDE inhibitor by simultaneous or successive or concomitant administration of antilipemics can probably be explained by the fact that antilipemics improve the disturbed endothelium function by generating nitrogen monoxide (NO) (Current Opinion in Lipidology, 1997, Vol. 8, pages 362-368 and Circulation 1998, 97, pages 1129-1135). As a neurotransmitter, nitrogen monoxide in turn is an important physiological factor for an erection to take place, since it increases the cGMP concentration, which finally results in a relaxation of the corpus cavernosum (International Journal of Impotence Research 1999, 11, pages 123-132 and 159-165).

According to the present invention, the antilipemic can be selected from the group consisting of:
HMG-CoA-reductase inhibitors
squalene synthase inhibitors,
bile acid absorption inhibitors (also referred to as “bile acid anion exchangers” or bile acid sequestrants),
fibric acid and its derivatives,
nicotinic acids and its analogs and also
ω3-fatty acids.

For further details about the antilipemics mentioned above, reference is in this context made to the article by Gilbert R. Thompson & Rissitaza P. Naoumova “New prospects for lipid-lowering drugs” in Exp. Opin. Invest. Drugs (1998), 7(5), pages 715-727, the entire content of which is hereby expressly incorporated by way of reference.

Among the antilipemics mentioned above, preference according to the invention is given to the HMG-CoA-reductase inhibitors. Here, the abbreviation “HMG-CoA” denotes “3-hydroxymethylglutaryl-coenzyme A”.

Among the HMG-CoA-reductase inhibitors, in turn, preference according to the invention is given, in particular, to the substance class of the vastatins—which, for the sake of simplicity, are in most cases referred to in the literature simply as “statins”.

Among the statins, in turn, particular preference according to the invention is given to
atorvastatin (commercially available under the name Lipitor® from Parke-Davis);
cerivastatin (commercially available under the name Lipobay® or Baycol® from Bayer);
fluvastatin (commercially available under the name Lescol® from Novartis);
lovastatin (commercially available under the name Mevacor® from Merck);
pravastatin (commercially available under the name Lipostat® from Bristol-Myers Squibb);
simvastatin (commercially available under the name Zocor® from Merck);
itavastatin (also called “nisvastatin”; NK-104; systematic name: [S-[R*,S*-(E)]]-7-[2-cyclopropyl-4-(4-fluorophenyl)-3-quinolinyl]-3,5-dihydroxy-6-heptenoic acid);
dalvastatin;
mevastatin;
dihydrocompactin;
compactin; and
(+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid;
   and their respective salts, hydrates, alkoxides, esters and tautomers,
   and among these with very particular preference atorvastatin, cerivastatin, fluvastatin, lovastatin, pravastatin, itavastatin, simvastatin and (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid and their respective salts, hydrates, alkoxides, esters and tautomers.

Among these, in turn, very particular preference is given to cerivastatin and atorvastatin and their respective salts, hydrates, alkoxides, esters and tautomers.

For further details about the statins mentioned above, reference is made to the discourse in Drugs of the Future 1994, 19(6), pages 537-541 and 1995, 20(6), page 611 and 1996, 21(6), page 642, the respective content of which is included herein in its entirety by way of reference.

For the purpose of the present invention, the term “salt” refers in each case to physiologically acceptable salts of the compounds in question: these can, for example, be salts with mineral acids, carboxylic acids or sulfonic acids, in particular with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid, or else mixed salts thereof. However, the salts can also be salts with customary bases, such as, for example, alkali metal salts (for example sodium or potassium salts), alkaline earth metal salts (for example calcium or magnesium salts) or ammonium salts, derived from ammonia or organic amines, such as, for example, diethylamine, triethylamine, ethyldiisopropylamine, procaine, dibenzylamine, N-methylmorpholine, dihydro-abietylamine, 1-ephenamine or methyl-piperidine, and also mixed salts thereof.

Examples of statin salts which can be used according to the invention are fluindostatin (the monosodium salt of fluvastatin); the monopotassium salt and the calcium salt of itavastatin; and the calcium salt of (+)-(3R,5S)-bis-(7-(4-(4-fluorophenyl)-6-isopropyl-2-(N-methyl-N-methane-sulfonylamino)-pyrimidin-5-yl)-3,5-dihydroxy-6(E)-heptenoic acid (“ZD 4522” or “S 4522” from Shionogi and AstraZeneca, respectively). Further examples of statin salts which can be used according to the invention are the monosodium and the monopotassium salts and also the calcium salts of cerivastatin, atorvastatin and pravastatin.

Further preferred HMG-CoA-reductase inhibitors are described in EP-A-0 325 130 and EP-A-0-491 226, both in the name of Bayer AG, the content of which is hereby included by way of reference. EP-A-0 325 130 provides substituted pyridines, and EP-A-0-491 226 describes substituted pyridyldihydroxyheptenoic acid derivatives and their salts, and among these in particular cerivastatin, which is particularly preferred according to the invention (claim 6 of EP-A-0-491 226).

Preference according to the invention is also given to the statins mentioned in WO-A-99/11263, the disclosure of which is included by way of reference.

Preference according to the invention is likewise given to the HMG-CoA-reductase inhibitors mentioned in the publication Bioorganic & Medicinal Chemistry, Vol. 5, No. 2, pages 437-444 (1997), the disclosure of which is hereby included in its entirety by way of reference.

A further review of HMG-CoA-reductase inhibitors can be found in Pharmazie in unserer Zeit, Vol. 28, No. 3, pages 147-1152 (1999).

Among the bile acid sequestrants mentioned above, preference according to the invention is given to cholestyramine (commercially available under the name Questran® from Bristol-Myers Squibb) and colestipol (commercially available under the name Colestid® from Pharmacia & Upjohn) (see also Exp. Opin. Invest. Drugs (1998), 7(5), pages 715-727).

Among the fibric acid derivatives mentioned above, preference according to the invention is given to ciprofibrate (commercially available under the name Modalim® from Sanofi Winthrop), fenofibrate (commercially available under the name Lipantil® from Fournier), gemfibrozil (commercially available under the name Lopid® from Parke-Davis), bezafibrate and chlofibrate (see also Exp. Opin. Invest. Drugs (1998), 7(5), pages 715-727).

Among the nicotinic acid analogs mentioned above, preference according to the invention is given to acipimox (commercially available under the name Olbetam® from Pharmacia & Upjohn) (see also Exp. Opin. Invest. Drugs (1998), 7(5), pages 715-727).

Among the ω3-fatty acids mentioned above, preference according to the invention is given to Maxepa (distributed by Seven Seas) (see also Exp. Opin. Invest. Drugs (1998), 7(5), pages 715-727).

Among the phosphodiesterase inhibitors, preference according to the invention is given in particular to cGMP PDE inhibitors. Among these, in turn, preference is given in particular to those which can be administered orally and, at the same time, also have good activity following oral administration.

Examples of cGMP PDE inhibitors which can be used and are preferred according to the invention are in particular the pyrazolopyrimidones described in EP-A-0 463 756, EP-A-0 526 004, WO-A-94/28902 and EP-B-0 702 555, the respective content of which is hereby included by way of reference. These are in particular compounds of the general formula below 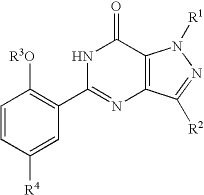
in which
R¹ represents: hydrogen; C₁-C₃-alkyl; C₁-C₃-perfluoroalkyl; or C₃-C₅-cycloalkyl;
R² denotes: hydrogen; C₁-C₆-alkyl, optionally substituted by C₃-C₆-cycloalkyl; C₁-C₃-perfluoroalkyl; or C₃-C₆-cycloalkyl;
R³ is: C₁-C₆-alkyl, optionally substituted by C₃-C₆-cycloalkyl; C₁-C₆-perfluoroalkyl; C₃-C₅-cycloalkyl; C₃-C₆alkenyl; or C₃-C₆-alkinyl;
R⁴ represents: C₁-C₄-alkyl, optionally substituted by OH, NR⁵R⁶, CN, CONR⁵R⁶ or CO₂R⁷; C₂-C₄-alkenyl, optionally substituted by CN, CONR⁵R⁶ or CO₂R⁷; C₂-C₄-alkanoyl, optionally substituted by NR⁵R⁶; (hydroxy)-C₂-C₄-alkyl, optionally substituted by NR⁵R⁶, (C₂-C₃-alkoxy)-C₁-C₂-alkyl, optionally substituted by OH or NR⁵R⁶, CO₂R⁷; halogen; NR⁵R⁶, NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl, each of which is optionally substituted by methyl;
R⁵ and R⁶ each independently of one another denote hydrogen or C₁-C₄-alkyl; or together with the nitrogen atom to which they are attached form a pyrrolidinyl, piperidino, morpholino, 4-N(R¹¹)-piperazinyl or imidazolyl group, where this group is optionally substituted by methyl or OH;
R⁷ is hydrogen or C₁-C₄-alkyl;
R⁸ represents C₁-C₃-alkyl, optionally substituted by NR⁵R⁶;
R⁹ and R¹⁰ together with the nitrogen atom to which they are attached form a pyrrolidinyl, piperidino, morpholino, 4-N(R¹²)-piperazinyl group, where this group is optionally substituted by C₁-C₄-alkyl, C₁-C₃-alkoxy, NR¹³R¹⁴ or CONR¹³R¹⁴;
R¹¹ denotes hydrogen, C₁-C₃-alkyl, optionally substituted by phenyl; (hydroxy)-C₂-C₃-alkyl; or C₁-C₄-alkanoyl;
R¹² is hydrogen, C₁-C₆-alkyl, (C₁-C₃-alkoxy)-C₂-C₆-alkyl; (hydroxy)-C₂-C₆-alkyl; (R¹³R¹⁴N)—C₂-C₆-alkyl; (R¹³R¹⁴NOC)—C₁-C₆-alkyl; CONR¹³R¹⁴; CSNR¹³R¹⁴, or C(NH)NR¹³R¹⁴; and
R¹³ and R¹⁴ each independently of one another represent hydrogen; C₁-C₄-alkyl; (C₁-C₃-alkoxy)-C₂-C₄-alkyl; or (hydroxy)-C₂-C₄-alkyl,
   and their respective salts, hydrates, alkoxides and tautomers.

Among these, very particular preference according to the invention is given to 5-[3-ethoxy-5-(4-methyl-1-piperazinylsulfonyl)-phenyl]-1-methyl-3-n-propyl-6,7-di-hydro-1H-pyrazolo-[4,3-d]-pyrimidin-7-one (other name: 5-[2-ethoxy-5-(4-methyl-1-piperazinyl-sulfonyl)-phenyl]-1-methyl-3-n-propyl-1,6-dihydro-7H-pyrazolo-[4,3-d]-pyrimidin-7-one, also known as “sildenafil”) and its salts, thus, for example, in particular the citrate salt, which is commercially available under the name Viagra™ (see in particular example 12 and compound 3 of claim 3 of EP-A-0 463 756 and also claim 6 of EP-B-0 702 555). cGMP PDE inhibitors which are likewise preferred according to the invention are the compounds described in WO-A-99/24433, the content of which is hereby included in its entirety by way of reference. The compounds in question are 2-phenyl-substituted imidazotriazinones of the general formula 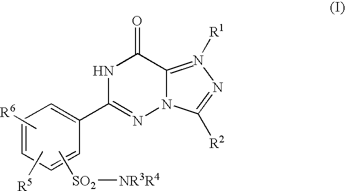
in which
R¹ represents hydrogen or straight-chain or branched alkyl having up to 4 carbon atoms;
R² represents straight-chain alkyl having up to 4 carbon atoms;
R³ and R⁴ are identical or different and represent hydrogen or represent straight-chain or branched alkenyl or alkoxy having in each case up to 8 carbon atoms, or
   represent a straight-chain or branched alkyl chain having up to 10 carbon atoms which is optionally interrupted by an oxygen atom and which is optionally mono- to polysubstituted by identical or different substituents from the group consisting of trifluoromethyl, trifluoromethoxy, hydroxyl, halogen, carboxyl, benzyloxycarbonyl, straight-chain or branched alkoxycarbonyl having up to 6 carbon atoms and/or by radicals of the formulae —SO₃H, -(A)ₐ-NR⁷R⁸, —O—CO—NR^{7′}R^{8′}, —S(O)_{b}—R⁹, —P(O)(OR¹⁰)(OR¹¹), 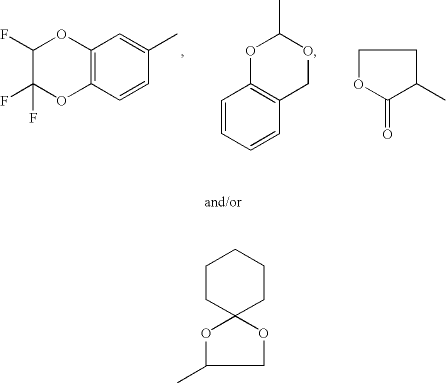
      in which
a and b are identical or different and represent a number 0 or 1,
A represents a radical CO or SO₂,
R⁷, R^{7′}, R⁸ and R^{8′} are identical or different and represent hydrogen, or represent cycloalkyl having 3 to 8 carbon atoms, aryl having 6 to 10 carbon atoms, a 5- to 6-membered unsaturated, partially unsaturated or saturated optionally benzo-fused heterocycle having up to 3 heteroatoms from the group consisting of S, N and O, where the abovementioned ring systems are optionally mono- to polysubstituted by identical or different substituents from the group consisting of hydroxyl, nitro, trifluoromethyl, trifluoromethoxy, carboxyl, halogen, straight-chain or branched alkoxy or alkoxycarbonyl having in each case up to 6 carbon atoms or by a group of the formula —(SO₂)_{c}—NR¹²R¹³,
   in which
   c represents a number 0 or 1,
   R¹² and R¹³ are identical or different and represent hydrogen or straight-chain or branched alkyl having up to 5 carbon atoms, or
R⁷, R^{7′}, R⁸ and R^{8′} represent straight-chain or branched alkoxy having up to 6 carbon atoms, or
   represent straight-chain or branched alkyl having up to 8 carbon atoms which is optionally mono- or polysubstituted by identical or different substituents from the group consisting of hydroxyl, halogen, aryl having 6 to 10 carbon atoms, straight-chain or branched alkoxy or alkoxycarbonyl having in each case up to 6 carbon atoms, or by a group of the formula —(CO)_{d}—NR¹⁴R¹⁵,
   in which
   R¹⁴ and R¹⁴ are identical or different and represent hydrogen or straight-chain or branched alkyl having up to 4 carbon atoms, and
   d represents a number 0 or 1, or
R⁷ and R⁸ and/or R^{7′} and R^{8′} together with the nitrogen atom form a 5- to 7-membered saturated heterocycle which may optionally contain a further heteroatom from the group consisting of S and O or a radical of the formula —NR¹⁶,
   in which
   R¹⁶ represents hydrogen, aryl having 6 to 10 carbon atoms, benzyl, a 5- to 7-membered aromatic or saturated heterocycle having up to 3 heteroatoms from the group consisting of S, N and O, which heterocycle is optionally substituted by methyl, or represents straight-chain or branched alkyl having up to 6 carbon atoms which is optionally substituted by hydroxyl,
R⁹ represents aryl having 6 to 10 carbon atoms, or represents straight-chain or branched alkyl having up to 4 carbon atoms,
R¹⁰ and R¹¹ are identical or different and represent hydrogen or straight-chain or branched alkyl having up to 4 carbon atoms,
   and/or the alkyl chain listed above under R³/R⁴ is optionally substituted by cycloalkyl having 3 to 8 carbon atoms, aryl having 6 to 10 carbon atoms or by a 5- to 7-membered partially unsaturated, saturated or unsaturated optionally benzo-fused heterocycle which may contain up to 4 heteroatoms from the group consisting of S, N; O or a radical of the formula —NR¹⁷,
   in which
R¹⁷ represents hydrogen, hydroxyl, formyl, trifluoromethyl, straight-chain or branched acyl or alkoxy having in each case up to 4 carbon atoms, or represents straight-chain or branched alkyl having up to 6 carbon atoms which is optionally mono- to polysubstituted by identical or different substituents from the group consisting of hydroxyl and straight-chain or branched alkoxy having up to 6 carbon atoms,
   and where aryl and the heterocycle are optionally mono- to polysubstituted by identical or different substituents from the group consisting of nitro, halogen, —SO₃H, straight-chain or branched alkyl or alkoxy having in each case up to 6 carbon atoms, hydroxyl, trifluoromethyl, trifluoromethoxy and/or by a radical of the formula —SO₂NR¹⁸R¹⁹,
   in which
   R¹⁸ and R¹⁹ are identical or different and represent hydrogen or straight-chain or branched alkyl having up to 6 carbon atoms, and/or
R³ or R⁴ represent a group of the formula —NR²⁰R²¹,
   in which
   R²⁰ and R²¹ have the meaning of R¹⁸ and R¹⁹ given above and are identical to or different from this meaning, and/or
R³ or R⁴ represent adamantyl, or
   represent radicals of the formulae 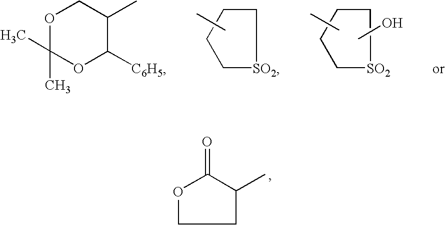
   or represent cycloalkyl having 3 to 8 carbon atoms, aryl having 6 to 10 carbon atoms or represent a 5- to 7-membered partially unsaturated, saturated or unsaturated optionally benzo-fused heterocycle which may contain up to 4 heteroatoms from the group consisting of S, N; O or a radical of the formula —NR²²,
      in which
R²² has the meaning of R¹⁶ given-above and is identical to or different from this meaning, or
   represents carboxyl, formyl or straight-chain or branched acyl having up to 5 carbon atoms,
      and where cycloalkyl, aryl and/or the heterocycle are optionally mono- to polysubstituted by identical or different substituents from the group consisting of halogen, triazolyl, trifluoromethyl, trifluoromethoxy, carboxyl, straight-chain or branched acyl or alkoxycarbonyl having in each case up to 6 carbon atoms, nitro, and/or by groups of the formulae —SO₃H, —OR²³, (SO₂)ₑNR²⁴R²⁵, —P(O)(OR²⁶)(OR²⁷),
      in which
e represents a number 0 or 1,
R²³ represents a radical of the formula 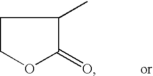
   represents cycloalkyl having 3 to 7 carbon atoms, or represents hydrogen or straight-chain or branched alkyl having up to 4 carbon atoms which is optionally substituted by cycloalkyl having 3 to 7 carbon atoms, benzyloxy, tetrahydropyranyl, tetrahydrofuranyl, straight-chain or branched alkoxy or alkoxycarbonyl having in each case up to 6 carbon atoms, carboxyl, benzyloxycarbonyl or phenyl which for its part may be mono- to polysubstituted by identical or different substituents from the group consisting of straight-chain or branched alkoxy having up to 4 carbon atoms, hydroxyl and halogen, and/or alkyl is optionally substituted by radicals of the formulae —CO—NR²⁸R²⁹ or —CO—R³⁰,
   in which.
   R²⁸ and R²⁹ are identical or different and represent hydrogen or straight-chain or branched alkyl having up to 8 carbon atoms, or
   R²⁸ and R²⁹ together with the nitrogen atom form a 5- to 7-membered saturated heterocycle which may optionally contain a further heteroatom from the group consisting of S and O, and
   R³⁰ represents phenyl or adamantyl,
R²⁴ and R²⁵ have the meaning of R¹⁸ and R¹⁹ given above and are identical to or different from this meaning,
R²⁶ and R²⁷ have the meaning of R¹⁰ and R¹¹ given above and are identical to or different from this meaning
   and/or cycloalkyl, aryl and/or the heterocycle are optionally substituted by straight-chain or branched alkyl having up to 6 carbon atoms which is optionally substituted by hydroxyl, carboxyl, by a 5- to 7-membered heterocycle having up to 3 heteroatoms from the group consisting of S, N and O or by groups of the formula —SO2-R³¹, P(O)(OR³²)(OR³³) or —NR³⁴R³⁵,
   in which
R³¹ is hydrogen or has the meaning of R⁹ given above and is identical to or different from this meaning,
   R³² and R³³ have the meaning of R¹⁰ and R¹¹ given above and are identical to or different from this meaning,
   R³⁴ and R³⁵ are identical or different and represent hydrogen or straight-chain or branched alkyl having up to 6 carbon atoms which is optionally substituted by hydroxyl or straight-chain or branched alkoxy having up to 4 carbon atoms, or
   R³⁴ and R³⁵ together with the nitrogen atom form a 5- to 6-membered saturated heterocycle which may contain a further heteroatom from the group consisting of S and O or a radical of the formula —NR³⁶,
      in which
      R³⁶ represents hydrogen, hydroxyl, straight-chain or branched alkoxycarbonyl having up to 7 carbon atoms or straight-chain or branched alkyl having up to 5 carbon atoms which is optionally substituted by hydroxyl, or
R³ and R⁴ together with the nitrogen atom form a 5- to 7-membered unsaturated or saturated or partially unsaturated optionally benzo-fused heterocycle which may optionally contain up to 3 heteroatoms from the group consisting of S, N, O or a radical of the formula —NR³⁷,
   in which
   R³⁷ represents hydrogen, hydroxyl, formyl, trifluoromethyl, straight-chain or branched acyl, alkoxy or alkoxycarbonyl having in each case up to 4 carbon atoms,
      or represents straight-chain or branched alkyl having up to 6 carbon atoms which is optionally mono- to polysubstituted by identical or different substituents from the group consisting of hydroxyl, trifluoromethyl, carboxyl, straight-chain or branched alkoxy or alkoxycarbonyl having in each case up to 6 carbon atoms or by groups of the formula -(D)_{f}—NR³⁸R³⁹, —CO—(CH₂)_{g}—O—CO—R⁴⁰, —CO—(CH₂)ₕ—OR⁴¹ or —P(O)(OR⁴²)(OR⁴³),
      in which
      g and h are identical or different and represent a number 1, 2, 3 or 4, and
      f represents a number 0 or 1,
      D represents a group of the formula —CO or —SO₂,
      R³⁸ and R³⁹ are identical or different and have the meaning of R⁷ and R⁸ given above,
      R⁴⁰ represents straight-chain or branched alkyl having up to 6 carbon atoms,
      R⁴¹ represents straight-chain or branched alkyl having up to 6 carbon atoms,
      R⁴² and R⁴³ are identical or different and represent hydrogen or straight-chain or branched alkyl having up to 4 carbon atoms, or
   R³⁷ represents a radical of the formula —(CO)ᵢ-E,
   in which
   represents a number 0 or 1,
   E represents cycloalkyl having 3 to 7 carbon atoms or benzyl, represents aryl having 6 to 10 carbon atoms or a 5- to 6-membered aromatic heterocycle having up to 4 heteroatoms from the group consisting of S, N and O, where the ring systems listed above are optionally mono- to polysubstituted by identical or different substituents from the group consisting of nitro, halogen, —SO₃H, straight-chain or branched alkoxy having up to 6 carbon atoms, hydroxyl, trifluoromethyl, trifluoromethoxy or by a radical of the formula —SO₂—NR⁴⁴R⁴⁵,
      in which
      R⁴⁴ and R⁴⁵ have the meaning of R¹⁸ and R¹⁹ given above and are identical to or different from this meaning, or
   E represents radicals of the formulae 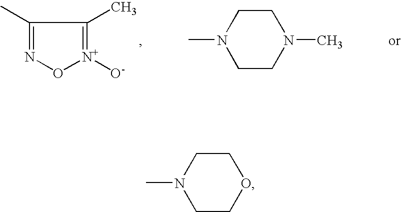
      and the heterocycle listed under R³ and R⁴, which is formed together with the nitrogen atom, is optionally mono- to polysubstituted by identical or different substituents, if appropriate also geminally, by hydroxyl, formyl, carboxyl, straight-chain or branched acyl or alkoxycarbonyl having in each case up to 6 carbon atoms, nitro and groups of the formulae —P(O)(OR⁴⁶)(OR⁴⁷), 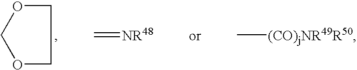
in which
R⁴⁶ and R⁴⁷ have the meaning of R¹⁰ and R¹¹ given above and are identical to or different from this meaning,
R⁴⁸ is hydroxyl or straight-chain or branched alkoxy having up to 4 carbon atoms,
j is a number 0 or 1, and
R⁴⁹ and R⁵⁰ are identical or different and have the meaning of R¹⁴ and R¹⁵ given above,
   and/or the heterocycle listed under R³ and R⁴, which is formed together with the nitrogen atom, is optionally substituted by straight-chain or branched alkyl having up to 6 carbon atoms which is optionally mono- to polysubstituted by identical or different substituents from the group consisting of hydroxyl, halogen, carboxyl, cycloalkyl or cycloalkyloxy having in each case 3 to 8 carbon atoms, straight-chain or branched alkoxy or alkoxycarbonyl having in each case up to 6 carbon atoms or by a radical of the formula —SO₃H, —NR⁵¹R⁵² or P(O)OR⁵³OR⁵⁴,
   in which
   R⁵¹ and R⁵² are identical or different and represent hydrogen, phenyl, carboxyl, benzyl or straight-chain or branched alkyl or alkoxy having in each case up to 6 carbon atoms,
   R⁵³ and R⁵⁴ are identical or different and have the meaning of R¹⁰ and R¹¹ given above,
   and/or the alkyl is optionally substituted by aryl having 6 to 10 carbon atoms which for its part may be mono- to polysubstituted by identical or different substituents from the group consisting of halogen, hydroxyl, straight-chain or branched alkoxy having up to 6 carbon atoms, or by a group of the formula —NR^{51′}R^{52′},
   in which
   R^{51′} and R^{52′} have the meaning of R⁵¹ and R⁵² given above and are identical to or different from this meaning,
   and/or the heterocycle listed under R³ and R⁴, which is formed together with the nitrogen atom, is optionally substituted by aryl having 6 to 10 carbon atoms or by a 5- to 7-membered saturated, partially unsaturated or unsaturated heterocycle having up to 3 heteroatoms from the group consisting of S, N and O, if appropriate also attached via an N-function, where the ring systems for their part may be substituted by hydroxyl or by straight-chain or branched alkyl or alkoxy having in each case up to 6 carbon atoms, or
R³ and R⁴ together with the nitrogen atom form radicals of the formulae 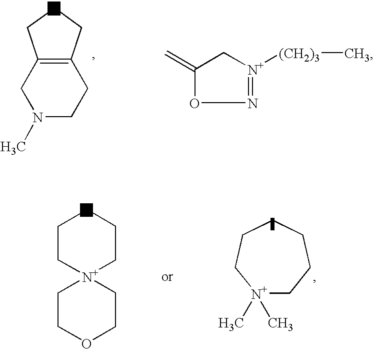
R⁵ and R⁶ are identical or different and represent hydrogen, straight-chain or branched alkyl having up to 6 carbon atoms, hydroxyl or represent straight-chain or branched alkoxy having up to 6 carbon atoms,
   and their respective salts, hydrates, alkoxides and tautomers.

Among the compounds mentioned in WO-A-99/24433, very particular preference according to the invention is given to 2-[2-ethoxy-5-(4-ethyl-piperazine-1-sulfonyl)-phenyl]-5-methyl-7-propyl-3H-imidazo[5,1-f][1,2,4]triazin-4-one (Ex. 19 of WO-A-99/24433) and its salts, such as, for example, the hydrochloride (Ex. 20 of WO-A-99/24433), in particular in the form of the trihydrate (Ex. 336 of WO-A-99/24433).

Further preferred PDE V inhibitors according to the invention are (a) zaprinast (Am. J. Physiol., Vol. 264, February 1993, pages H419-H422; The Journal of Urology, Vol. 147, pages 1650-1655 (June 1992); The New England Journal of Medicine, Vol. 326(2), pages 90-94 (9 January 1992)); (b) propentofylline (JP-A-03/044324); and (c) pentoxifylline (Postgraduate Medicine, Vol. 93, No. 3, Impotence, 15 Feb. 1993, pages 65-72; J.A.G.S., 41, pages 363-366, 1993).

Further PDE V inhibitors which are preferred according to the invention are disclosed in the following publications, the contents of which are included in their entirety by way of reference:
5-substituted pyrazolo-[4,3-d]-pyrimidin-7-one according to EP-A-0 201 188;
griseolic acid derivatives according to EP-A-0 214 708 and EP-A-0 319 050;
2-phenylpurinone derivatives according to EP-A-0 293 063;
phenylpyridone derivatives according to EP-A-0 347 027;
fused pyrimidine derivatives according to EP-A-0 347 146;
condensed pyrimidine derivatives according to EP-A-0 349 239;
pyrimidopyrimidine derivatives according to EP-A-0 351 058;
purine compounds according to EP-A-0 352 960;
quinazoline derivatives according to EP-A-0 371 731;
phenylpyrimidone derivatives according to EP-A-0 395 328;
imidazoquinoxalinone derivatives or their aza analogs according to EP-A-0 400 583;
phenylpyrimidone derivatives according to EP-A-0 400 799;
phenylpyridone derivatives according to EP-A-0 428 268;
pyrimidopyrimidine derivatives according to EP-A-0 442 204;
4-aminoquinazoline derivatives according to EP-A-0 579 496;
4,5-dihydro-4-oxo-pyrrolo-[1,2-a]-quinoxaline derivatives and their aza analogs according to EP-A-0 584 487;
polycyclic guanine derivatives according to WO-A-91/19717;
nitrogen-containing heterocycles according to WO-A-93/07124;
polycyclic 2-benzyl-guanine derivatives according to WO-A-94/19351;
quinazoline derivatives according to U.S. Pat. No. 4,060,615;
6-heterocyclyl-pyrazolo-[3,4-d]-pyrimidin-4-ones according to U.S. Pat. No. 5,294,612;
benzimidazoles according to JP-A 5-222000;
cycloheptimidazole according to European Journal of Pharmacology 1994, 251, page 1;
N-containing heterocycles according to WO-A-94/22855.

Further PDE inhibitors which are preferred and can be used according to the invention are:
tetracyclic derivatives according to WO-A-95/19978;
pyrazolopyrimidine derivatives according to EP-A-0 636 626;
4-aminopyrimidine derivatives according to EP-A-0 640 599;
imidazoquinazoline derivatives according to EP-A-0 668 280;
quinazoline compounds according to EP-A-0 669 324;
4-aminoquinazoline derivatives according to U.S. Pat. No. 5,436,233.

Further PDE inhibitors which are likewise preferred and can be used according to the invention are the compounds according to EP-A-0 579 496; WO-A-93/07124; U.S. Pat. No. 5,294,612 and WO-A-94/22855, the contents of which are hereby included by way of reference.

Examples of PDE inhibitors from the publications mentioned above which are particularly preferred according to the invention are the following compounds:
1,3-dimethyl-5-benzylpyrazolo-[4,3-d]-pyrimidin-7-one (preparation according to EP-A-0 201 188, Example 1);
2-(2-propoxyphenyl)-6-purinone (preparation according to EP-A-0 293 063, Example 1);
6-(2-propoxyphenyl)-1,2-dihydro-2-oxopyridine-3-carboxamide (preparation according to EP-A-0 347 027, Example 2);
2-(2-propoxyphenyl)-pyrido-[2,3-d]-pyrimid-4(3H)-one (preparation according to EP-A-0 347 146, Example 1);
7-methylthio-4-oxo-2-(2-propoxyphenyl)-3,4-dihydropyrimido-[4,5-d]-pyrimidine (preparation according to EP-A-0 351 058, Example 1);
6-hydroxy-2-(2-propoxyphenyl)-pyrimidine4-carboxamide (preparation according to EP-A-0 395 328, Example 15);
1-ethyl-3-methylimidazo-[1,5-a]-quinoxalin-4(5H)-one (preparation according to EP-A-0 400 583);
4-phenylmethylarnino-6-chloro-2-(1-imidazolyl)-quinoxaline (preparation according to EP-A-0 579 496, Example 5(c));
5-ethyl-8-[3-(N-cyclohexyl-N-methylcarbamyl)-propyloxy]-4,5-dihydro-4-oxo-pyrido-[3,2-e]-pyrrolo-[1,2-a]-pyrazine (preparation according to EP-A-0 584 487, Example 1);
5′-methyl-3′-(phenylmethyl)-spiro-[cyclopentane-1,7′(8′H)-(3′H)-imidazo[2,1-b]-purin]-4′(5′H)-one (preparation according to WO-A-91/19717, Example 9A3);
1-[6-chloro-4-(3,4-methylenedioxybenzyl)-aminoquinazolin-2-yl)-piperidine-4-carboxylic acid (preparation according to WO-A-93/97124);
(6aR,9aS)-2-(4-trifluoromethylphenyl)-methyl-5-methyl-3,4,5,6a,7,8,9,9a-octahydrocyclopent-[4,5]-imidazo-[2,1-b]-purin-4-one (preparation according to WO-A-94/19351, Example 14);
1-tert-butyl-3-phenylmethyl-6-(4-pyridyl)-pyrazolo-[3,4-d]-pyrimid-4-one (preparation according to U.S. Pat. No. 5,294,612, Example 90);
1-cyclopentyl-3-methyl-6-(4-pyridyl)-4,5-dihydro-1H-pyrazolo-[3,4-d]-pyrimid-4-one (preparation according to U.S. Pat. No. 5,294,612, Example 83);
2-butyl-1-(2-chlorobenzyl)-6-ethoxycarbonylbenzimidazole (preparation according to JP-A-5-222000);
2-(4-carboxypiperidino)-4-(3,4-methylenedioxybenzyl)-amino-6-nitroquinazoline (preparation according to WO-A-94/22855, Example II);
2-phenyl-8-ethoxycycloheptimidazole (KT2-734);
1-[6-chloro-4-(3,4-methyldioxybenzyl)-aminoquinazolin-2-yl]-piperidine-4-carboxylic acid (preparation according to WO-A-93/07124);
(6aR,9aS)-2-(4-trifluoromethylphenyl)-methyl-5-methyl-3,4,5,6a,7,8,9,9a-octahydrocyclopent-[4,5]-imidazo-[2,1-b]-purin-4-one (preparation according to WO-A-94/1935 1, Example 14);
1-tert-butyl-3-phenylmethyl-6-(4-pyridyl)-pyrazolo-[3,4-d]-pyrimid-4-one (preparation according to U.S. Pat. No. 5,294,612, Example 90);
1-cyclopentyl-3-methyl-6-(4-pyridyl-4,5-dihydro-1H-pyrazolo-[3,4-d]-pyrimid-4-one (preparation according to U.S. Pat. No. 5,294,612, Example 83);
2-(4-carboxypiperidino)-4-(3,4-methylenedioxybenzyl)-amino-6-nitro-quinazoline (preparation according to WO-A-94/22855, Example II).

Further cGMP PDE inhibitors which can be used according to the invention are the following compounds:
pyrazolopyrimidine derivatives according to EP-A-0 636 626;
4-aminopyrimidine derivatives according to EP-A-0 640 599;
imidazoquinazoline derivatives according to WO-A-95/06648;
anthranilic acid derivatives according to WO-A-95/18097;
4-aminoquinazoline derivatives according to U.S. Pat. No. 5,436,233;
tetracyclic derivatives according to WO-A-95/19978;
imidazoquinazoline derivatives according to EP-A-0 668 280;
quinazoline compounds according to EP-A-0 669 324.

Further PDE inhibitors which are preferred according to the invention, in particular cGMP PDE inhibitors, are disclosed in the following publications, whose contents are hereby included by way of reference:
quinolone derivatives according to WO-A-98/53819;
pyrazolo[4,3-d]-pyrimidine derivatives according to EP-A-911333;
pyrazolopyrimidones according to WO-A-98/49166;
compounds according to WO-A-96/16644;
bicyclic heterocycles according to WO-A-96/16657;
pyrazolopyrimidones according to WO-A-93/06104;
pyrazolopyrimidones according to WO-A-93/07149;
quinazolines according to WO-A-93/12093;
purinones according to WO-A-94/00453;
pyridopyrimidinones according to WO-A-94/05661;
compounds according to U.S. Pat. No. 5,272,147 and U.S. Pat. No. 5,250,534; and
quinazolinones according to WO-A-93/12095.

Further PDE inhibitors which are preferred according to the invention, in particular cGMP PDE inhibitors, are selected from the group consisting of:
4-(3-chloro-4-methoxybenzyl)amino-1-(4-hydroxypiperidino)-6-phthalazine-carbonitrile and its salts, tautomers, alkoxides and hydrates, in particular 4-(3-chloro-4-methoxybenzyl)-amino-1-(4-hydroxypiperidino)-6-phthalazine-carbonitrile hydrochloride;
1-[4-[(1,3-benzodioxol-5-ylmethyl)amino]-6-chloro-2-quinazolyl]-4-piperidine-carboxylic acid and its salts, tautomers, alkoxides and hydrates, in particular the sodium salt;
4-bromo-6-[3-(4-chlorophenyl)propoxy]-5-[(3-piperidinylmethyl)amino]-3(2H)-pyridazinone and its salts, tautomers, alkoxides and hydrates, in particular 4-bromo-6-[3-(4-chlorophenyl)propoxy]-5-[(3-piperidinylmethyl)-amino]-3(2H)-pyridazinone hydrochloride.

Further PDE inhibitors which are preferred according to the invention, in particular cGMP PDE inhibitors, are disclosed in the following publications, whose contents are hereby included by way of reference:
carbazole derivatives and their salts according to WO-A-99/21831 (Fujisawa);
dihydropyrazolopyrimidinone derivatives according to WO-A-98/40384 (BAYER) and DE-A-197 09 877 (BAYER);
purine and pyrazolopyrimidine derivatives according to DE-A-197 09 126 (BAYER);
isoquinolinone derivatives according to WO-A-98/38168 (Tanabe);
substituted azaindole compounds according to JP-A-10120681 (Fujisawa);
indolizine compounds according to JP-A-10120680 (Fujisawa);
(amino-thieno-pyrimidinyl)-heterocyclic acid derivatives according to DE-A-196 44 228 (Merck) and WO-A-98/17668 (Merck);
2-amino-thiophene-3-carboxamide derivatives according to DE-A-196 42 451 (Merck) and WO-A-98/16521 (Merck);
pyridocarbazole derivatives according to WO-A-97/45427 (Mochida);
purinone derivatives according to EP-A-771799 (BAYER);
N-benzyl-3-indenylacetamide derivatives according to WO-A-99/31065 (Baverstock/Cell Pathways/Univ. Arizona State);
pyridocarbazole compounds according to WO-A-99/26946 (Mochida);
thienopyrimidine derivatives according to DE-A-1 97 52 952 (Merck) and WO-A-99/28325 (Merck);
indole derivatives according to WO-A-98/15530 (Fujisawa);
pyrazolopyridazinones according to WO-A-98/14448 (Kyorin);
phenylalkylthienopyrimidine derivatives according to DE-A-196 32 423 (Merck) and WO-A-98/06722 (Merck);
compounds according to WO-A-96/21435 (Euroceltique);
dihydropyrazolopyrroles according to WO-A-95/19362 (Pfizer);
compounds according to WO-A-95/00516 (Euroceltique).

In addition to the two active compound components A and B mentioned above, the combination preparation according to the invention may comprise any further active compounds, provided these compounds are not contraindicated for this area of indication and do not adversely affect the action of the PDE inhibitor and the antilipemic.

These further optionally present active compounds, like the active compound components A and B, can be present either as a true mixture together with A and/or B or else spatially separated therefrom. They can be administered concomitantly or simultaneously or successively with the active compound component(s) A and/or B.

The further optionally present active compounds of the combination preparation according to the invention include, for example:
further active compounds which improve the capability to maintain an erection and which do not belong to the class of the PDE inhibitors, such as, for example: α-adrenergic antagonists, such as, for example, yohimbine or Vasomax® from Zonagen; or else substances as mentioned in WO-A-98/52569, the content of which is hereby included by way of reference; or prostaglandine-E1; or seretonin antagonists;
active compounds from the cardiovascular field of indication;
active compounds from the CNS and cerebral field of indication;
vitamins;
minerals;
trace elements.

Suitable administration forms for the administration of the two active compound components A and B (and any other active compounds present) are in each case all customary administration forms. The administration is preferably carried out orally, perlingually, sublingually, nasally, transdermally, buccally, intravenously, rectally, inhalatively or parenterally. The administration is preferably carried out orally, sublingually or nasally. Very particular preference is given to oral administration.

In the case of spatially separated and/or successive administration, the two active compound components A and B can also be administered in different administration forms.

The two active compound components A and B—together or spatially separated—can in each case be converted in a manner known per se into customary formulations such as tablets including coated tablets, pills, granules, aerosols, syrups, emulsions, suspensions and solutions, using inert, non-toxic, pharmaceutically suitable carriers or solvents. In this case, the therapeutically active components A and B should in each case be present in a concentration from approximately 0.5 to 90% by weight of the total mixture, i.e. in amounts which are sufficient in order to achieve the dosage range indicated.

The formulations are prepared, for example, by extending the two active compound components A and B with solvents and/or carriers, if appropriate using emulsifying agents and/or dispersing agents, it being possible, for example if the diluent used is water, to use organic solvents as auxiliary solvents, if appropriate.

For use in humans, in the case of oral administration, dosages of from 0.001 to 50 mg/kg, preferably from 0.001 mg/kg to 20 mg/kg, in particular from 0.001 to 10 mg/kg, of body weight, particularly preferably from 0.001 mg/kg to 5 mg/kg, of the respective active compound component A or B are administered to obtain effective and useful results.

In spite of this, it may be necessary, if appropriate, to depart from the amounts mentioned, namely depending on the body weight and/or on the type of administration route, on the individual response toward the combination preparation, the manner of its formulation and the time or interval at which administration takes place. Thus, in some cases it may be adequate to manage with less than the abovementioned minimum amount, while in other cases the upper limit mentioned must be exceeded.

In the case of the administration of relatively large amounts, it may be advisable to divide these into several individual administrations over the course of the day.

The present invention is illustrated by the embodiment below in a purely exemplary manner, and by no means limited thereby.

### EXAMPLE

In a placebo-controlled study, twelve adult male rabbits of the animal strain HsdHHL Watanables having a body weight of 3-5 kg, which, owing to breeding, suffer from hypercholesterolemia and arteriosclerosis, were treated with a selective cGMP PDE inhibitor (3 mg/kg, intravenously).

The animals had free access to water and, for 2 hours per day, to feed. The animals were kept in a 10/14 hour day/night rhythm (light on from 8 am onwards), and the ambient temperature was 22 to 24° C.

The selective cGMP PDE inhibitor used was 2-[2-ethoxy-5-(4-ethyl-piperazine-1-sulfonyl)-phenyl]-5-methyl-7-propyl-3H-imidazo[5,1-f][1,2,4]triazin-4-one hydrochloride trihydrate (Ex. 336 of WO-A-99/24433).

For intravenous injection, the PDE inhibitor was dissolved in physiological saline (0.9% by volume).

For seven days prior to the treatment with the PDE inhibitor, i.e. for 7 successive days, an antilipemic, i.e. cerivastatin sodium (i.e. the monosodium salt of cerivastatin) was administered subcutaneously to six of the twelve rabbits (1 mg/kg of body weight, cerivastatin sodium dissolved in physiological saline).

Instead of this, a physiological saline was administered subcutaneously to the other six rabbits (control).

In each case 1 day and 3 days after the 7-day pretreatment with cerivastatin sodium had ended, the six rabbits which had been pretreated with cerivastatin sodium were given the PDE inhibitor.

The other six rabbits, which had not been pretreated with cerivastatin sodium, were given the PDE inhibitor 1 day after the 7-day subcutaneous administration of the saline (control) had ended.

The erection was evaluated by measuring the length of the protruding penis using a vernier caliper. The measurement was carried out in each case 5, 10, 15, 30, 45, 60 and 120 minutes after the administration of the PDE inhibitor. To this end, the animals were in each case removed from the cage, held by neck hair and hind legs, turned onto their back and measured. Under rest conditions, i.e. in the non-erect state, the penis of the rabbit is not visible in the pubic region and completely covered by the penis skin.

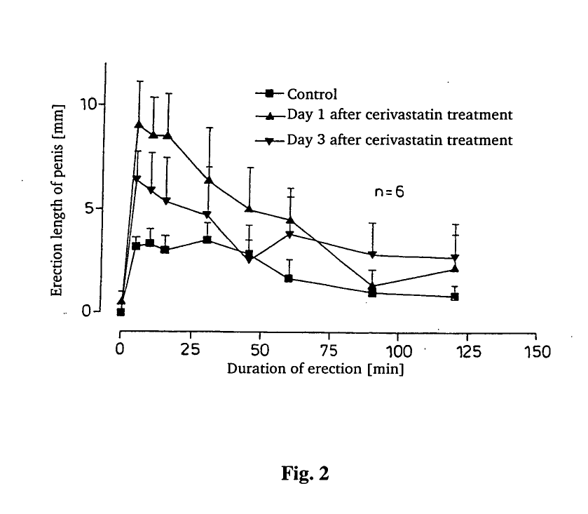

As illustrated by the attached FIG. 2, the six rabbits which had been pretreated with the antilipemic cerivastatin sodium (upper and middle curves) showed, after administration of the PDE inhibitor, surprisingly a considerably more pronounced erection than the six rabbits which had not been pretreated with the antilipemic (lower curve).

Comparison of the upper and middle curves of FIG. 2 also shows that the enhanced capability to sustain an erection in the pretreated animals decreased again once the treatment with the antilipemic was stopped. Thus, the erection 1 day after the cerivastatin sodium pretreatment had ended was still more pronounced than even 3 days after the cerivastatin sodium pretreatment had ended. This too confirms that the surprising improvement in the activity of the PDE inhibitor is caused by the antilipemic.

As shown by the experiment, even a pretreatment with the antilipemic which is initiated shortly before the administration of the PDE inhibitor causes, entirely unexpectedly, an enhanced PDE-inhibitor-triggered erection. In this manner, the activity of the PDE inhibitor is surprisingly improved, since, in view of the short pretreatment interval with the antilipemic, no (significant) improvement in the hypercholesterolemia, not to mention the arteriosclerosis, had occurred.

Without wishing to adhere to a certain theory, the synergistic effect of the PDE inhibitor together with the antilipemic can possibly be explained by an improved endothelium function, as already discussed above in the general description. This synergistic effect, however, was totally unforeseeable for the person skilled in the art and therefore has to be considered to be entirely surprising.

Thus, the embodiment confirms in an impressive manner the improved PDE-inhibitory action of the PDE inhibitor by the antilipemic administered in combination therewith.

However, the person skilled in the art will be familiar with numerous further embodiments when practicing the present invention, without being outside of the scope of the present invention.

1. A combination preparation, comprising as pharmaceutically active ingredients at least one active compound component A and at least one active compound component B, characterized in that the active compound component A is a PDE inhibitor, preferably a cGMP PDE inhibitor, and the active compound component B is an antilipemic.

2. The combination preparation as claimed in claim 1 for treating sexual dysfunction in men or women.

3. The combination preparation as claimed in claim 2 for treating erectile dysfunction.

4. The combination preparation as claimed in any of claims 1 to 3, characterized in that the two active compound components A and B are used either simultaneously or successively.

5. The combination preparation as claimed in any of claims 1 to 4, characterized in that the active compound components A and B are present as a functional unit, in particular in the form of a mixture, a mix or a blend.

6. The combination preparation as claimed in any of claims 1 to 4, characterized in that the active compound components A and B are (spatially) separated, in particular as a kit-of-parts.

7. The combination preparation as claimed in any of claims I to 6, characterized in that the antilipemic (active compound component B) is selected from the group consisting of (a) HMG-CoA-reductase inhibitors; (b) squalene synthase inhibitors; (c) bile acid sequestrants; (d) fibric acid and its derivatives; (e) nicotinic acid and its analogs; (f) ω3-fatty acids.

8. The combination preparation as claimed in claim 7, characterized in that the antilipemic (active compound component B) is an HMG-CoA-reductase inhibitor and is in particular selected from the group of the statins, preferably from the group consisting of atorvastatin, cerivastatin, fluvastatin, lovastatin, pravastatin, itavastatin, simvastatin and (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid, and their respective salts, hydrates, alkoxides, esters and tautomers.

9. The combination preparation as claimed in claim 8, characterized in that the antilipemic (active compound component B) is atorvastatin or its salt, hydrate, alkoxide, ester and tautomer.

10. The combination preparation as claimed in claim 8, characterized in that the antilipemic (active compound component B) is cerivastatin or its salt, hydrate, alkoxide, ester and tautomer.

11. The combination preparation as claimed in any of claims 1 to 10, characterized in that the PDE inhibitor (active compound component A) is a cGMP PDE inhibitor and is in particular selected from the group consisting of pyrazolopyrimidones of the general formula below 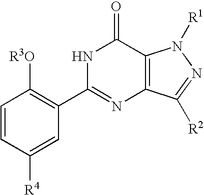
in which
R¹ represents hydrogen; C₁-C₃-alkyl; C₁-C₃-perfluoroalkyl; or C₃-C₅-cycloalkyl;
R² denotes hydrogen; C₁-C₆-alkyl, optionally substituted by C₃-C₆-cycloalkyl; C₁-C₃-perfluoroalkyl; or C₃-C₆-cycloalkyl;
R³ is C₁-C₆-alkyl, optionally substituted by C₃-C₆-cycloalkyl; C₁-C₆-perfluoroalkyl, C₃-C₅-cycloalkyl; C₃-C₆alkenyl; or C₃-C₆-alkinyl;
R⁴ represents C₁-C₄-alkyl, optionally substituted by OH, NR⁵R⁶, CN, CONR⁵R⁶ or CO₂R⁷; C₂-C₄-alkenyl, optionally substituted by CN, CONR⁵R⁶ or CO₂R⁷; C₂-C₄-alkanoyl, optionally substituted by NR⁵R⁶; (hydroxy)-C₂-C₄-alkyl, optionally substituted by NR⁵R⁶, (C₂-C₃-alkoxy)-C₁-C₂-alkyl, optionally substituted by OH or NR⁵R⁶, CO₂R⁷; halogen; NR⁵R⁶, NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl, each of which is optionally substituted by methyl;
R⁵ and R⁶ each independently of one another denote hydrogen or C₁-C₄-alkyl; or together with the nitrogen atom to which they are attached form a pyrrolidinyl, piperidino, morpholino, 4-N(R¹¹)-piperazinyl or imidazolyl group, where this group is optionally substituted by methyl or OH;
R⁷ is hydrogen or C₁-C₄-alkyl;
R⁸ represents C₁-C₃-alkyl, optionally substituted by NR⁵R⁶;
R⁹ and R¹⁰ together with the nitrogen atom to which they are attached form a pyrrolidinyl, piperidino, morpholino, 4-N(R¹²)-piperazinyl group, where this group is optionally substituted by C₁-C₄-alkyl, C₁-C₃-alkoxy, NR¹³R¹⁴ or CONR¹³R¹⁴;
R¹¹ denotes hydrogen, C₁-C₃-alkyl, optionally substituted by phenyl; (hydroxy)-C₂-C₃-alkyl; or C₁-C₄-alkanoyl;
R¹² is hydrogen, C₁-C₆-alkyl, (C₁-C₃-alkoxy)-C₂-C₆-alkyl; (hydroxy)-C₂-C₆-alkyl; (R¹³R¹⁴N)—C₂-C₆-alkyl; (R¹³R¹⁴NOC)—C₁-C₆-alkyl; CONR¹³R¹⁴; CSNR¹³R¹⁴, or C(NH)NR¹³R¹⁴; and
R¹³ and R¹⁴ each independently of one another represent hydrogen; C₁-C₄-alkyl; (C₁-C₃-alkoxy)-C₂-C₄-alkyl; or (hydroxy)-C₂-C₄-alkyl,
and their respective salts, hydrates, alkoxides and tautomers.

12. The combination preparation as claimed in any of claims 1 to 10, characterized in that the PDE inhibitor (active compound component A) is a cGMP PDE inhibitor and is in particular selected from the group consisting of 2-phenyl-substituted imidazotriazinones of the general formula 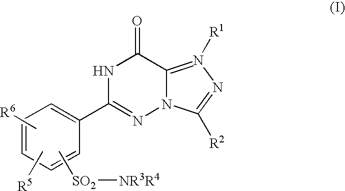
in which
R¹ represents hydrogen or straight-chain or branched alkyl having up to 4 carbon atoms;
R² represents straight-chain alkyl having up to 4 carbon atoms;
R³ and R⁴ are identical or different and represent hydrogen or represent straight-chain or branched alkenyl or alkoxy having in each case up to 8 carbon atoms, or
represent a straight-chain or branched alkyl chain having up to 10 carbon atoms which is optionally interrupted by an oxygen atom and which is optionally mono- to polysubstituted by identical or different substituents from the group consisting of trifluoromethyl, trifluoromethoxy, hydroxyl, halogen, carboxyl, benzyloxycarbonyl, straight-chain or branched alkoxycarbonyl having up to 6 carbon atoms and/or by radicals of the formulae —SO₃H, -(A)ₐ-NR⁷R⁸, —O—CO—NR^{7′}R^{8′}, —S(O)_{b}—R⁹, —P(O)(OR¹⁰)(OR¹¹), 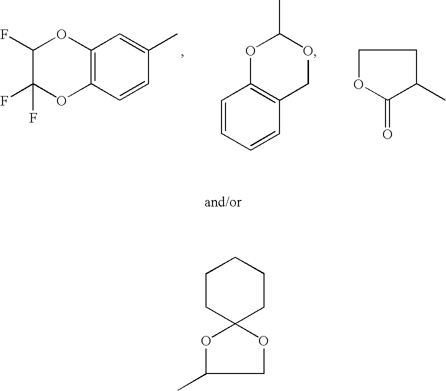
in which
a and b are identical or different and represent a number 0 or 1,
A represents a radical CO or SO₂,
R⁷, R^{7′}, R⁸ and R^{8′} are identical or different and represent hydrogen, or represent cycloalkyl having 3 to 8 carbon atoms, aryl having 6 to 10 carbon atoms, a 5- to 6-membered unsaturated, partially unsaturated or saturated optionally benzo-fused heterocycle having up to 3 heteroatoms from the group consisting of S, N and O, where the abovementioned ring systems are optionally mono- to polysubstituted by identical or different substituents from the group consisting of hydroxyl, nitro, trifluoromethyl, trifluoromethoxy, carboxyl, halogen, straight-chain or branched alkoxy or alkoxycarbonyl having in each case up to 6 carbon atoms or by a group of the formula —(SO₂)_{c}—NR¹²R¹³,
in which
c represents a number 0 or 1,
R¹² and R¹³ are identical or different and represent hydrogen or straight-chain or branched alkyl having up to 5 carbon atoms, or
R⁷, R^{7′}, R⁸ and R^{8′} represent straight-chain or branched alkoxy having up to 6 carbon atoms, or
represent straight-chain or branched alkyl having up to 8 carbon atoms which is optionally mono- or polysubstituted by identical or different substituents from the group consisting of hydroxyl, halogen, aryl having 6 to 10 carbon atoms, straight-chain or branched alkoxy or alkoxycarbonyl having in each case up to 6 carbon atoms, or by a group of the formula —(CO)_{d}—NR¹⁴R¹⁵,
in which
R¹⁴ and R¹⁵ are identical or different and represent hydrogen or straight-chain or branched alkyl having up to 4 carbon atoms, and
d represents a number 0 or 1, or
R⁷ and R⁸ and/or R^{7′} and R^{8′} together with the nitrogen atom form a 5- to 7-membered saturated heterocycle which may optionally contain a further heteroatom from the group consisting of S and O or a radical of the formula —NR¹⁶,
in which
R¹⁶ represents hydrogen, aryl having 6 to 10 carbon atoms, benzyl, a 5- to 7-membered aromatic or saturated heterocycle having up to 3 heteroatoms from the group consisting of S, N and O, which heterocycle is optionally substituted by methyl, or represents straight-chain or branched alkyl having up to 6 carbon atoms which is optionally substituted by hydroxyl,
R⁹ represents aryl having 6 to 10 carbon atoms, or represents straight-chain or branched alkyl having up to 4 carbon atoms,
R¹⁰ and R¹¹ are identical or different and represent hydrogen or straight-chain or branched alkyl having up to 4 carbon atoms,
and/or the alkyl chain listed above under R³/R⁴ is optionally substituted by cycloalkyl having 3 to 8 carbon atoms, aryl having 6 to 10 carbon atoms or by a 5- to 7-membered partially unsaturated, saturated or unsaturated optionally benzo-fused heterocycle which may contain up to 4 heteroatoms from the group consisting of S, N; O or a radical of the formula —NR¹⁷,
in which
R¹⁷ represents hydrogen, hydroxyl, formyl, trifluoromethyl, straight-chain or branched acyl or alkoxy having in each case up to 4 carbon atoms, or represents straight-chain or branched alkyl having up to 6 carbon atoms which is optionally mono- to polysubstituted by identical or different substituents from the group consisting of hydroxyl and straight-chain or branched alkoxy having up to 6 carbon atoms,
and where aryl and the heterocycle are optionally mono- to polysubstituted by identical or different substituents from the group consisting of nitro, halogen, —SO₃H, straight-chain or branched alkyl or alkoxy having in each case up to 6 carbon atoms, hydroxyl, trifluoromethyl, trifluoromethoxy and/or by a radical of the formula —SO₂NR¹⁸R¹⁹,
in which
R¹⁸ and R¹⁹ are identical or different and represent hydrogen or straight-chain or branched alkyl having up to 6 carbon atoms, and/or
R³ or R⁴ represent a group of the formula —NR²⁰R²¹,
in which
R²⁰ and R²¹ have the meaning of R¹⁸ and R¹⁹ given above and are identical to or different from this meaning, and/or
R³ or R⁴ represent adamantyl, or
represent radicals of the formulae 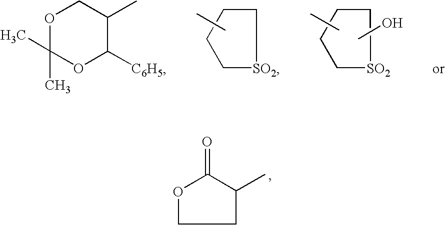
or represent cycloalkyl having 3 to 8 carbon atoms, aryl having 6 to 10 carbon atoms or represent a 5- to 7-membered partially unsaturated, saturated or unsaturated optionally benzo-fused heterocycle which may contain up to 4 heteroatoms from the group consisting of S, N; O or a radical of the formula —NR²²,
in which
R²² has the meaning of R given above and is identical to or different from this meaning, or
represents carboxyl, formyl or straight-chain or branched acyl having up to 5 carbon atoms,
and where cycloalkyl, aryl and/or the heterocycle are optionally mono- to polysubstituted by identical or different substituents from the group consisting of halogen, triazolyl, trifluoromethyl, trifluoromethoxy, carboxyl, straight-chain or branched acyl or alkoxycarbonyl having in each case up to 6 carbon atoms, nitro, and/or by groups of the formulae —SO₃H, —OR²³, (SO₂)ₑNR²⁴R²⁵, —P(O)(OR²⁶)(OR²⁷),
in which
e represents a number 0 or 1,
R²³ represents a radical of the formula 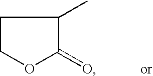
represents cycloalkyl having 3 to 7 carbon atoms, or represents hydrogen or straight-chain or branched alkyl having up to 4 carbon atoms which is optionally substituted by cycloalkyl having 3 to 7 carbon atoms, benzyloxy, tetrahydropyranyl, tetrahydrofuranyl, straight-chain or branched alkoxy or alkoxycarbonyl having in each case up to 6 carbon atoms, carboxyl, benzyloxycarbonyl or phenyl which for its part may be mono- to polysubstituted by identical or different substituents from the group consisting of straight-chain or branched alkoxy having up to 4 carbon atoms, hydroxyl and halogen,
and/or alkyl is optionally substituted by radicals of the formulae —CO—NR²⁸R²⁹ or —CO—R³⁰,
in which
R²⁸ and R²⁹ are identical or different and represent hydrogen or straight-chain or branched alkyl having up to 8 carbon atoms, or
R²⁸ and R²⁹ together with the nitrogen atom form a 5- to 7-membered saturated heterocycle which may optionally contain a further heteroatom from the group consisting of S and O, and
R³⁰ represents phenyl or adamantyl,
R²⁴ and R²⁵ have the meaning of R¹⁸ and R¹⁹ given above and are identical to or different from this meaning,
R²⁶ and R²⁷ have the meaning of R¹⁰ and R¹¹ given above and are identical to or different from this meaning
and/or cycloalkyl, aryl and/or the heterocycle are optionally substituted by straight-chain or branched alkyl having up to 6 carbon atoms which is optionally substituted by hydroxyl, carboxyl, by a 5- to 7-membered heterocycle having up to 3 heteroatoms from the group consisting of S, N and O or by groups of the formula —SO2—R31, P(O)(OR³²)(OR³³) or —NR³⁴R³⁵,
in which
R³¹ is hydrogen or has the meaning of R⁹ given above and is identical to or different from this meaning,
R³² and R³³ have the meaning of R¹⁰ and R¹¹ given above and are identical to or different from this meaning,
R³⁴ and R³⁵ are identical or different and represent hydrogen or straight-chain or branched alkyl having up to 6 carbon atoms which is optionally substituted by hydroxyl or straight-chain or branched alkoxy having up to 4 carbon atoms, or
R³⁴ and R³⁵ together with the nitrogen atom form a 5- to 6-membered saturated heterocycle which may contain a further heteroatom from the group consisting of S and O or a radical of the formula —NR³⁶,
in which
R³⁶ represents hydrogen, hydroxyl, straight-chain or branched alkoxycarbonyl having up to 7 carbon atoms or straight-chain or branched alkyl having up to 5 carbon atoms which is optionally substituted by hydroxyl, or
R³ and R⁴ together with the nitrogen atom form a 5- to 7-membered unsaturated or saturated or partially unsaturated optionally benzo-fused heterocycle which may optionally contain up to 3 heteroatoms from the group consisting of S, N, O or a radical of the formula —NR³⁷,
in which
R³⁷ represents hydrogen, hydroxyl, formyl, trifluoromethyl, straight-chain or branched acyl, alkoxy or alkoxycarbonyl having in each case up to 4 carbon atoms,
or represents straight-chain or branched alkyl having up to 6 carbon atoms which is optionally mono- to polysubstituted by identical or different substituents from the group consisting of hydroxyl, trifluoromethyl, carboxyl, straight-chain or branched alkoxy or alkoxycarbonyl having in each case up to 6 carbon atoms or by groups of the formula —(D)_{f}—NR³⁸R³⁹, —CO—(CH₂)_{g}—O—CO—R⁴⁰, —CO—(CH₂)ₕ—OR⁴¹ or —P(O)(OR⁴²)(OR⁴³),
in which
g and h are identical or different and represent a number 1, 2, 3 or 4, and
f represents a number 0 or 1,
D represents a group of the formula —CO or —SO₂,
R³⁸ and R³⁹ are identical or different and have the meaning of R⁷ and R⁸ given above,
R⁴⁰ represents straight-chain or branched alkyl having up to 6 carbon atoms,
R⁴¹ represents straight-chain or branched alkyl having up to 6 carbon atoms,
R⁴² and R⁴³ are identical or different and represent hydrogen or straight-chain or branched alkyl having up to 4 carbon atoms, or
R³⁷ represents a radical of the formula —(CO)ᵢ-E,
in which
i represents a number 0 or 1,
E represents cycloalkyl having 3 to 7 carbon atoms or benzyl, represents aryl having 6 to 10 carbon atoms or a 5- to 6-membered aromatic heterocycle having up to 4 heteroatoms from the group consisting of S, N and O, where the ring systems listed above are optionally mono- to polysubstituted by identical or different substituents from the group consisting of nitro, halogen, —SO₃H, straight-chain or branched alkoxy having up to 6 carbon atoms, hydroxyl, trifluoromethyl, trifluoromethoxy or by a radical of the formula —SO₂—NR⁴⁴R⁴⁵,
in which
R⁴⁴ and R⁴⁵ have the meaning of R¹⁸ and R¹⁹ given above and are identical to or different from this meaning, or
E represents radicals of the formulae 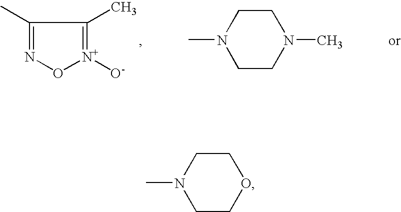
and the heterocycle listed under R³ and R⁴, which is formed together with the nitrogen atom, is optionally mono- to polysubstituted by identical or different substituents, if appropriate also geminally, by hydroxyl, formyl, carboxyl, straight-chain or branched acyl or alkoxycarbonyl having in each case up to 6 carbon atoms, nitro and groups of the formulae —P(O)(OR⁴⁶)(OR⁴⁷), 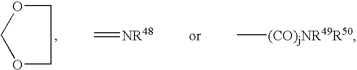
in which
R⁴⁶ and R⁴⁷ have the meaning of R¹⁰ and R¹¹ given above and are identical to or different from this meaning,
R⁴⁸ is hydroxyl or straight-chain or branched alkoxy having up to 4 carbon atoms,
j is a number 0 or 1, and
R⁴⁹ and R⁵⁰ are identical or different and have the meaning of R¹⁴ and R¹⁵ given above,
and/or the heterocycle listed under R³ and R⁴, which is formed together with the nitrogen atom, is optionally substituted by straight-chain or branched alkyl having up to 6 carbon atoms which is optionally mono- to polysubstituted by identical or different substituents from the group consisting of hydroxyl, halogen, carboxyl, cycloalkyl or cycloalkyloxy having in each case 3 to 8 carbon atoms, straight-chain or branched alkoxy or alkoxycarbonyl having in each case up to 6 carbon atoms or by a radical of the formula —SO₃H, —NR⁵¹R⁵² or P(O)OR⁵³OR⁵⁴,
in which
R⁵¹ and R⁵² are identical or different and represent hydrogen, phenyl, carboxyl, benzyl or straight-chain or branched alkyl or alkoxy having in each case up to 6 carbon atoms, R⁵³ and R⁵⁴ are identical or different and have the meaning of R¹⁰ and R¹¹ given above,
and/or the alkyl is optionally substituted by aryl having 6 to 10 carbon atoms which for its part may be mono- to polysubstituted by identical or different substituents from the group consisting of halogen, hydroxyl, straight-chain or branched alkoxy having up to 6 carbon atoms, or by a group of the formula —NR^{51′}R^{52′},
in which
R^{51′} and R^{52′} have the meaning of R⁵¹ and R⁵² given above and are identical to or different from this meaning,
and/or the heterocycle listed under R³ and R⁴, which is formed together with the nitrogen atom, is optionally substituted by aryl having 6 to 10 carbon atoms or by a 5- to 7-membered saturated, partially unsaturated or unsaturated heterocycle having up to 3 heteroatoms from the group consisting of S, N and O, if appropriate also attached via an N-function, where the ring systems for their part may be substituted by hydroxyl or by straight-chain or branched alkyl or alkoxy having in each case up to 6 carbon atoms, or
R³ and R⁴ together with the nitrogen atom form radicals of the formulae 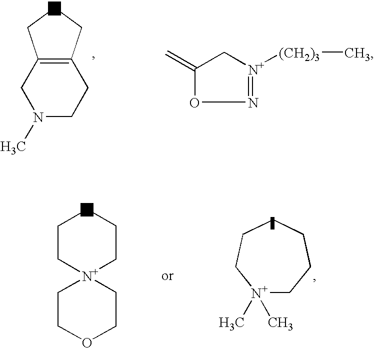
R⁵ and R⁶ are identical or different and represent hydrogen, straight-chain or branched alkyl having up to 6 carbon atoms, hydroxyl or represent straight-chain or branched alkoxy having up to 6 carbon atoms.
and their respective salts, hydrates, alkoxides and tautomers.

13. The combination preparation as claimed in any of claims 1 to 10, characterized in that the PDE inhibitor (active compound component A) is a cGMP PDE inhibitor and in particular is selected from the group consisting of (a) 5-[2-ethoxy-5-(4-methyl-1-piperazinylsulfonyl)-phenyl]-1-methyl-3-n-propyl-1,6-dihydro-7H-pyrazolo-[4,3-d]-pyrimidin-7-one (sildenafil) and its salts, hydrates, alkoxides and tautomers; and (b) 2-[2-ethoxy-5-(4-ethyl-piperazine-1-sulfonyl)-phenyl]-5-methyl-7-propyl-3H-imidazo[5,1-f]-[1,2,4]-triazin-4-one and its salts, hydrates, alkoxides and tautomers.

14. The combination preparation as claimed in claim 13, in that the PDE inhibitor (active compound component A) is 5-[2-ethoxy-5-(4-methyl-1-piperazinylsulfonyl)-phenyl]-1-methyl-3-n-propyl-1,6-dihydro-7H-pyrazolo-[4,3-d]-pyrimidin-7-one citrate (sildenafil citrate, Viagra™) or 2-[2-ethoxy-5-(4-ethylpiperazine-1-sulfonyl)-phenyl]-5-methyl-7-propyl-3H-imidazo[5,1-f][1,2,4]triazin-4-one hydrochloride trihydrate.

15. The use of antilipemics for enhancing the activity of PDE inhibitors, in particular cGMP PDE inhibitors.

16. The use as claimed in claim 15 in the treatment of sexual dysfunction in men and women, in particular in the treatment of erectile dysfunction.

17. The use as claimed in claim 15 or 16, characterized in that the antilipemic and the PDE inhibitor are used either simultaneously or else successively.

18. The use as claimed in any of claims 15 to 17, characterized in that the antilipemic and the PDE inhibitor are present as a functional unit, in particular in the form of a mixture, a mix or a blend.

19. The use as claimed in any of claims 15 to 17, characterized in that the antilipemic and the PDE inhibitor are present (spatially) separated, in particular as a kit-of-parts.

20. The use as claimed in any of claims 15 to 19, characterized in that the antilipemic is selected from the compounds defined in claims 7 to 10.

21. The use as claimed in any of claims 15 to 20, characterized in that the PDE inhibitor is selected from the compounds defined in claims 11 to 14.
